(19) United States
(12) Patent Application Publication
(10) Pub. No.: US 2019/0054238 A1
(43) Pub. Date: Feb. 21, 2019

(54) MULTIFUNCTIONAL ASSISTANT TREATMENT ROBOT

(71) Applicant: JINAN Youke Medical Technology Co., Ltd, Jinan City (CN)

(72) Inventor: Baofa YU, Jinan City (CN)

(21) Appl. No.: 15/902,940

(22) Filed: Feb. 22, 2018

(30) Foreign Application Priority Data
Aug. 17, 2017 (CN) 201710708674.0

(51) Int. Cl. A61M 5/20; A61B 17/132; A61M 21/02

(52) U.S. Cl. CPC A61M 5/20; A61M 2021/0027; A61M 21/02; A61B 17/132

(57) ABSTRACT

The present invention discloses a multifunctional assistant treatment robot, comprising a top plate, wherein the bottom of the top plate is provided with a first mounting groove into which a first pushrod motor is fixedly mounted with its output shaft being connected with a puncture needle, and the bottom of the top plate is welded with a second vertical plate, the bottom of the top plate is further provided with an inclined groove, an inclined plate is slidably connected onto a top inner wall of the inclined groove, the first pushrod motor is located between the second vertical plate and the inclined plate, a treatment bed is provided below the first mounting groove, and a foam plate is placed at the top of the treatment bed and is located below the puncture needle.

### CROSS REFERENCE TO RELATED APPLICATIONS

This application claims priority to Chinese Patent Application No. 201710708674.0, entitled “MULTIFUNCTIONAL ASSISTANT TREATMENT ROBOT,” which was filed on Aug. 17, 2017, the contents of which is hereby incorporated by reference in its entirety for all purposes.

### TECHNICAL FIELD

The present invention relates to the technical field of treatment robots, in particular to a multifunctional assistant treatment robot.

### BACKGROUND

At present, treatment increasingly tends to be intelligent. Now, treatment can be performed on a patient through an assistant treatment robot, thereby greatly reducing the working intensity of medical workers and improving the working efficiency of treatment for the patient. For example, an assistant treatment robot for facial paralysis disease disclosed in Chinese Application No. 201610940397.1 can automatically realize a function of massaging a face of a patient suffering from facial paralysis to promote facial blood circulation, meanwhile a medicine atomizing device is provided and the assistant treatment effect for a facial paralysis disease can be obviously improved through applying permeable medication. However, the patient suffering from facial paralysis sometimes needs assistant injection treatment, while the existing assistant treatment robot does not have autonomous bleeding stopping performance. Besides, the assistant treatment robot sometimes cannot successfully perform the injection in one time, mostly because nerves of the patient are high-strung such that muscles of the part on which injection is performed are in a high-strung state. Therefore, the inventors propose a multifunctional assistant treatment robot for solving the above-mentioned problems.

### SUMMARY

Aiming at the technical problems mentioned in the Background portion, the present invention provides a multifunctional assistant treatment robot.

The multifunctional assistant treatment robot provided by the present invention comprises a top plate, wherein the bottom of the top plate is provided with a first mounting groove into which a first pushrod motor is fixedly mounted with its output shaft being connected with a puncture needle, and the bottom of the top plate is welded with a second vertical plate, the bottom of the top plate is further provided with an inclined groove, an inclined plate is slidably connected onto a top inner wall of the inclined groove, the first pushrod motor is located between the second vertical plate and the inclined plate, a treatment bed is provided below the first mounting groove, and a foam plate is placed at the top of the treatment bed and is located below the puncture needle.

Preferably, the puncture needle comprises an injection rod, an injection tube and an injection needle, the output shaft of the first pushrod motor is connected with the injection rod, the bottom of the injection rod extends into the injection tube and is welded with a sealing plate which is slidably connected with an inner wall of the injection tube, an inner wall of one side of the inclined groove away from the first mounting groove is provided with a second mounting groove into which a second pushrod motor is fixedly mounted with its output shaft being welded with a pushing plate, one side of the inclined plate away from the first mounting groove is provided with a sliding groove, one side of the pushing plate close to the first mounting groove extends into the sliding groove and is slidably connected with inner walls of the sliding groove, inner walls of two sides of the sliding groove are provided with rectangular grooves, two sides of the pushing plate are welded with sliding blocks which are slidably connected with inner walls of the rectangular grooves, the bottom of the inclined plate is welded with a first vertical plate, the bottom of the first vertical plate extends below the top plate and is welded with a first horizontal plate, one side of the bottom of the first horizontal plate close to the first pushrod motor is provided with a third mounting groove into which a driving motor is fixedly mounted with its output shaft being welded with a rotating shaft, a bottom end of the rotating shaft is welded with two symmetrically arranged fixed plates, the sides of the two fixed plates close to each other are provided with a fourth mounting groove into which a third pushrod motor is fixedly mounted with its output shaft being welded with two moving rods, the ends of the two moving rods close to each other are welded with arc-shaped blocks, the sides of the two arc-shaped blocks close to each other are in contact with each other, and a same alcohol swab is fixedly mounted on inner sides of the two arc-shaped blocks and is located above the foam plate.

Preferably, the bottom of the second vertical plate is welded with a second horizontal plate, one side of the second horizontal plate close to the puncture needle is provided with a rectangular groove, one side of the puncture needle close to the second horizontal plate is welded with a rectangular block which is slidably connected with an inner wall of the rectangular groove, the bottom of the second horizontal plate is provided with a fifth mounting groove in which a fourth pushrod motor is mounted with its output shaft being welded with a pressing plate, and the bottom of the pressing plate is welded with a massage plate which is located above the foam plate.

Preferably, the arc-shaped blocks are located on one side of the puncture needle away from the second vertical plate, and the arc-shaped blocks are located above the foam plate.

Preferably, the two arc-shaped blocks are symmetrically arranged, the sides of the two arc-shaped blocks close to each other are provided with sealing gaskets, and the alcohol swab is slidably connected with inner sides of the sealing gaskets.

Preferably, the friction force between the sealing plate and the inner wall of the injection tube is greater than the friction force between the rectangular block and the inner wall of the rectangular groove.

Preferably, the bottom of the second horizontal plate is further provided with a sixth mounting groove in which a music player is fixedly mounted, and the sixth mounting groove is located on one side of the fifth mounting groove away from the puncture needle.

Preferably, the top of the fourth pushrod motor is welded with a mounting plate, four corners of the bottom of the mounting plate are provided with bolt holes into which bolts are threadedly connected, and the fourth pushrod motor is fixedly mounted on a top inner wall of the fifth mounting groove through the bolt holes and the bolts on the mounting plate.

As compared with the prior art, the present invention has the following beneficial effects: with the cooperation of the top plate, the first mounting groove, the first pushrod motor, the injection rod, the injection tube, the sealing plate, the injection needle, the treatment bed, the foam plate, the inclined groove, the second mounting groove, the second pushrod motor, the inclined plate, the sliding grooves, the pushing plate, the rectangular grooves, the sliding blocks, the first vertical plate, the first horizontal plate, the third mounting groove, the driving motor, the rotating shaft, the fixed plates, the fourth mounting grooves, the third pushrod motors, the moving rods, the arc-shaped blocks and the alcohol swab, the operation of using the alcohol swab to wipe the body of a patient and the change of the alcohol swab are facilitated, and the operation of stopping bleeding of a wound of the patient can be effectively assisted; and with the cooperation of the second vertical plate, the second horizontal plate, the rectangular groove, the rectangular block, the fifth mounting groove, the fourth pushrod motor, the pressing plate, the massage plate, the sixth mounting groove and the music player, nerves of the patient can be enabled to be in a relaxed state, so as to facilitate the injection treatment performed by the assistant treatment robot on the patient.

The present invention is highly practical, wherein the inclined plate and the arc-shaped blocks facilitate the operation of using an alcohol swab to wipe the body of a patient and the change of the alcohol swab, which can also effectively assist the operation of stopping bleeding of a wound of the patient; and the massage plate and music player can enable nerves of the patient to be in a relaxed state, so as to facilitate the injection treatment performed by the assistant treatment robot on the patient.

### BRIEF DESCRIPTION OF THE DRAWINGS

FIG. 1 illustrates a structural schematic view of a multifunctional assistant treatment robot provided by the present invention;

FIG. 2 illustrates a structural schematic view of part A of a multifunctional assistant treatment robot provided by the present invention;

FIG. 3 illustrates a structural schematic view of part B of a multifunctional assistant treatment robot provided by the present invention;

FIG. 4 illustrates a structural schematic side view of a pushing plate of a multifunctional assistant treatment robot provided by the present invention;

FIG. 5 illustrates a structural schematic bottom view of a rotating shaft of a multifunctional assistant treatment robot provided by the present invention.

In the drawings: 1—top plate; 2—first mounting groove; 3—first pushrod motor; 4—injection rod; 5—injection tube; 6—sealing plate; 7—injection needle; 8—treatment bed; 9—foam plate; 10—inclined groove; 11—second mounting groove; 12—second pushrod motor; 13—inclined plate; 14—sliding groove; 15—pushing plate; 16—rectangular groove; 17—sliding block; 18—first vertical plate; 19—first horizontal plate; 20—third mounting groove; 21—driving motor; 22—rotating shaft; 23—fixed plate; 24—fourth mounting groove; 25—third pushrod motor; 26—moving rod; 27—arc—shaped block; 28—alcohol swab; 29—second vertical plate; 30—second horizontal plate; 31—rectangular groove; 32—rectangular block; 33—fifth mounting groove; 34—fourth pushrod motor; 35—pressing plate; 36—massage plate; 37—sixth mounting groove; 38—music player.

### DETAILED DESCRIPTION OF THE INVENTION

The present invention will be further described below in combination with specific embodiments.

### Embodiments

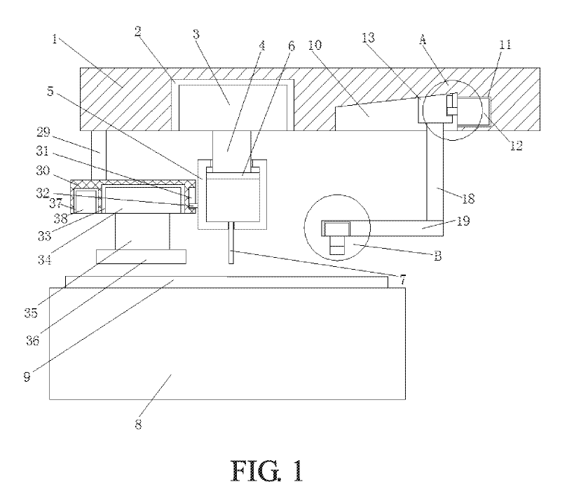

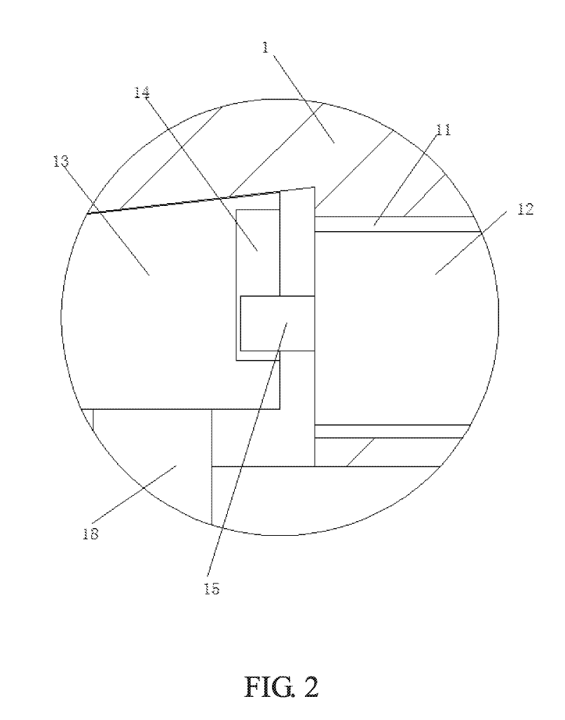

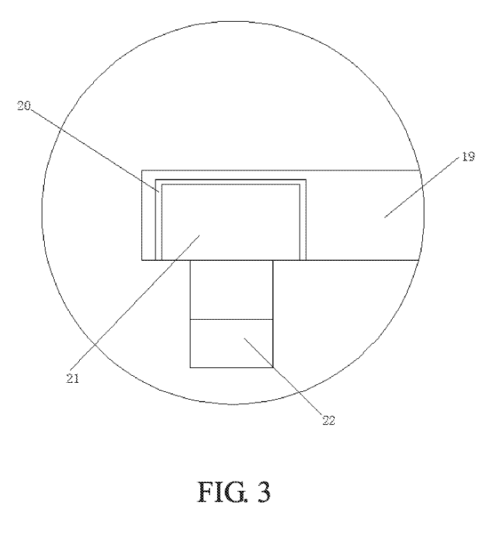

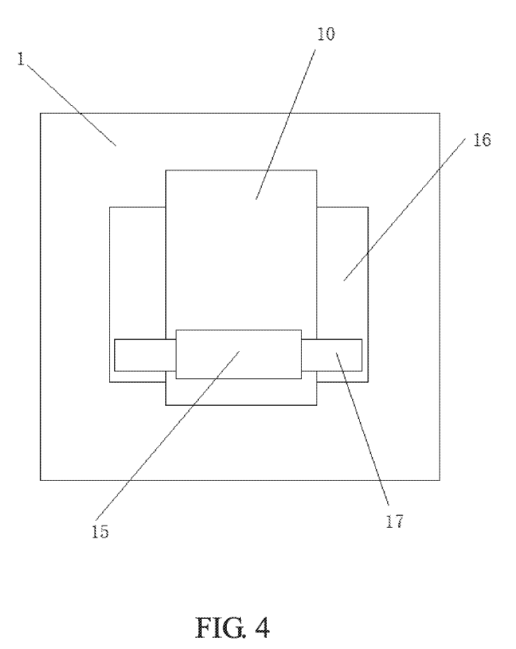

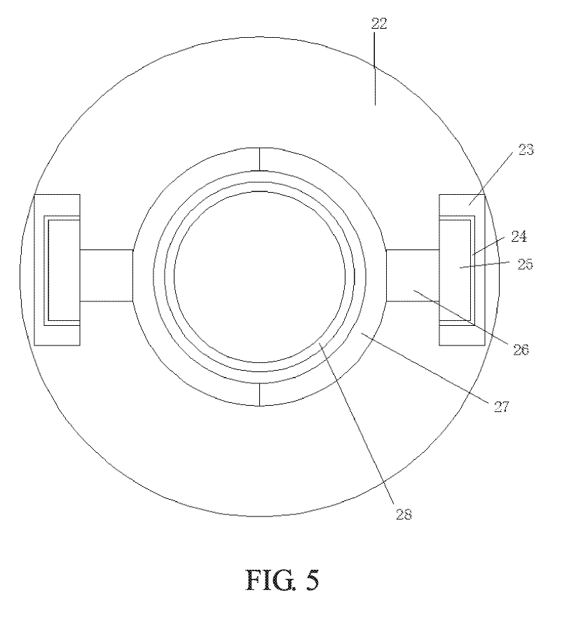

Referring to FIGS. 1-5, a multifunctional assistant treatment robot comprises a top plate 1, and the bottom of the top plate 1 is provided with a first mounting groove 2 into which a first pushrod motor 3 is fixedly mounted with its output shaft being connected with a puncture needle. The bottom of the top plate 1 is welded with a second vertical plate 29. The bottom of the top plate 1 is further provided with an inclined groove 10, an inclined plate 13 is slidably connected onto a top inner wall of the inclined groove 10, and the first pushrod motor 3 is located between the second vertical plate 29 and the inclined plate 13. A treatment bed 8 is provided below the first mounting groove 2, and a foam plate 9 is placed at the top of the treatment bed 8 and is located below the puncture needle. The puncture needle comprises an injection rod 4, an injection tube 5 and an injection needle 7. The output shaft of the first pushrod motor 3 is connected with the injection rod 4, and the bottom of the injection rod 4 extends into the injection tube 5 and is welded with a sealing plate 6 which is slidably connected with an inner wall of the injection tube 5. An inner wall of one side of the inclined groove 10 away from the first mounting groove 2 is provided with a second mounting groove 11 into which a second pushrod motor 12 is fixedly mounted with its output shaft being welded with a pushing plate 15. One side of the inclined plate 13 away from the first mounting groove 2 is provided with a sliding groove 14. One side of the pushing plate 15 close to the first mounting groove 2 extends into the sliding groove 14 and is slidably connected with inner walls of the sliding groove 14. Inner walls of two sides of the sliding groove 14 are provided with rectangular grooves 16, and two sides of the pushing plate 15 are welded with sliding blocks 17 which are slidably connected with inner walls of the rectangular grooves 16. The bottom of the inclined plate 13 is welded with a first vertical plate 18, and the bottom of the first vertical plate 18 extends below the top plate 1 and is welded with a first horizontal plate 19. One side of the bottom of the first horizontal plate 19 close to the first pushrod motor 3 is provided with a third mounting groove 20 into which a driving motor 21 is fixedly mounted with its output shaft being welded with a rotating shaft 22, and a bottom end of the rotating shaft 22 is welded with two symmetrically arranged fixed plates 23. The sides of the two fixed plates 23 close to each other are provided with a fourth mounting groove 24 into which a third pushrod motor 25 is fixedly mounted with its output shaft being welded with two moving rods 26. The ends of the two moving rods 26 close to each other are welded with arc-shaped blocks 27, and the sides of the two arc-shaped blocks 27 close to each other are in contact with each other. A same alcohol swab 28 is fixedly mounted on inner sides of the two arc-shaped blocks 27 and is located above the foam plate 9. The bottom of the second vertical plate 29 is welded with a second horizontal plate 30. One side of the second horizontal plate 30 close to the puncture needle is provided with a rectangular groove 31, and one side of the puncture needle close to the second horizontal plate 30 is welded with a rectangular block 32 which is slidably connected with an inner wall of the rectangular groove 31. The bottom of the second horizontal plate 30 is provided with a fifth mounting groove 33 in which a fourth pushrod motor 34 is mounted with its output shaft being welded with a pressing plate 35. The bottom of the pressing plate 35 is welded with a massage plate 36 which is located above the foam plate 9. With the cooperation of the top plate 1, the first mounting groove 2, the first pushrod motor 3, the injection rod 4, the injection tube 5, the sealing plate 6, the injection needle 7, the treatment bed 8, the foam plate 9, the inclined groove 10, the second mounting groove 11, the second pushrod motor 12, the inclined plate 13, the sliding grooves 14, the pushing plate 15, the rectangular grooves 16, the sliding blocks 17, the first vertical plate 18, the first horizontal plate 19, the third mounting groove 20, the driving motor 21, the rotating shaft 22, the fixed plates 23, the fourth mounting grooves 24, the third pushrod motors 25, the moving rods 26, the arc-shaped blocks 27 and the alcohol swab 28, the operation of using the alcohol swab to wipe the body of a patient and the change of the alcohol swab are facilitated, and the operation of stopping bleeding of a wound of the patient can be effectively assisted. With the cooperation of the second vertical plate 29, the second horizontal plate 30, the rectangular groove 31, the rectangular block 32, the fifth mounting groove 33, the fourth pushrod motor 34, the pressing plate 35, the massage plate 36, the sixth mounting groove 37 and the music player 38, nerves of the patient can be enabled to be in a relaxed state, so as to facilitate the injection treatment performed by the assistant treatment robot on the patient. The present invention is highly practical, wherein the inclined plate 13 and the arc-shaped blocks 27 facilitate the operation of using an alcohol swab 28 to wipe the body of a patient and the change of the alcohol swab 28, which can also effectively assist the operation of stopping bleeding of a wound of the patient; and the massage plate 36 and music player 38 can enable nerves of the patient to be in a relaxed state, so as to facilitate the injection treatment performed by the assistant treatment robot on the patient.

In this embodiment, the arc-shaped blocks 27 are located on one side of the puncture needle away from the second vertical plate 29, and the arc-shaped blocks 27 are located above the foam plate 9. The two arc-shaped blocks 27 are symmetrically arranged, and the sides of the two arc-shaped blocks 27 close to each other are provided with sealing gaskets, and the alcohol swab 28 is slidably connected with inner sides of the sealing gaskets. The friction force between the sealing plate 6 and the inner wall of the injection tube 5 is greater than the friction force between the rectangular block 32 and the inner wall of the rectangular groove 31. The bottom of the second horizontal plate 30 is further provided with a sixth mounting groove 37 in which a music player 38 is fixedly mounted, and the sixth mounting groove 37 is located on one side of the fifth mounting groove 33 away from the puncture needle. The top of the fourth pushrod motor 34 is welded with a mounting plate, and four corners of the bottom of the mounting plate are provided with bolt holes into which bolts are threadedly connected, and the fourth pushrod motor 34 is fixedly mounted on a top inner wall of the fifth mounting groove 33 through the bolt holes and the bolts on the mounting plate. With the cooperation of the top plate 1, the first mounting groove 2, the first pushrod motor 3, the injection rod 4, the injection tube 5, the sealing plate 6, the injection needle 7, the treatment bed 8, the foam plate 9, the inclined groove 10, the second mounting groove 11, the second pushrod motor 12, the inclined plate 13, the sliding grooves 14, the pushing plate 15, the rectangular grooves 16, the sliding blocks 17, the first vertical plate 18, the first horizontal plate 19, the third mounting groove 20, the driving motor 21, the rotating shaft 22, the fixed plates 23, the fourth mounting grooves 24, the third pushrod motors 25, the moving rods 26, the arc-shaped blocks 27 and the alcohol swab 28, the operation of using the alcohol swab 28 to wipe the body of the patient and the change of the alcohol swab 28 are facilitated and the operation of stopping bleeding of the wound of the patient can be effectively assisted. With the cooperation of the second vertical plate 29, the second horizontal plate 30, the rectangular groove 31, the rectangular block 32, the fifth mounting groove 33, the fourth pushrod motor 34, the pressing plate 35, the massage plate 36, the sixth mounting groove 37 and the music player 38, the nerves of the patient can be enabled to be in a relaxed state, so as to facilitate the injection treatment performed by the assistant treatment robot on the patient. The present invention is highly practical, wherein the inclined plate 13 and the arc-shaped blocks 27 facilitate the operation of using an alcohol swab 28 to wipe the body of a patient and the change of the alcohol swab 28, which can also effectively assist the operation of stopping bleeding of a wound of the patient; and the massage plate 36 and music player 38 can enable nerves of the patient to be in a relaxed state, so as to facilitate the injection treatment performed by the assistant treatment robot on the patient.

In this embodiment, when the nerves of the patient are in a high-strung state before injection treatment, the music player 38 is turned on, and the fourth pushrod motor 34 is started up. The fourth pushrod motor 34 drives the pressing plate 35 to move, the pressing plate 35 drives the massage plate 36 to move, and the massage plate 36 massages the body of the patient such that the patient can be relaxed, so as to improve the injection success rate of the assistant treatment robot. After the nerves of the patient are relaxed, the second pushrod motor 12 is started up, and the output shaft of the second pushrod motor 12 drives the pushing plate 15 to move such that the pushing plate 15 slides on the inner walls of the sliding grooves 14. The pushing plate 15 drives the sliding blocks 17 to move such that the sliding blocks 17 slide on the inner walls of the sliding grooves 16. Simultaneously, the pushing plate 15 pushes the inclined plate 13 to move such that the inclined plate 13 slides on the top inner wall of the inclined groove 10. The inclined plate 13 drives the first vertical plate 18 to move and the first vertical plate 18 drives the first horizontal plate 19 to move. When the inclined plate 13 is in contact with the inner wall of one side of the inclined groove 10 close to the first mounting groove 2, the alcohol swab 28 is exactly in contact with the body of the patient. The second pushrod motor 12 is shut down, the driving motor 21 is started up, the output shaft of the driving motor 21 drives the rotating shaft 22 to rotate, and the rotating shaft 22 drives the alcohol swab 28 to rotate such that the alcohol swab 28 fully wipes the part of the body of the patient on which the injection needs to be performed. After the wiping operation is completed, the driving motor 21 is shut down and the second pushrod motor 12 is started up. When the inclined plate 13 moves to an initial position, the second pushrod motor 12 is shut down, meanwhile the first pushrod motor 3 is started up and the first pushrod motor 3 drives the injection rod 4 to move. Since the friction force between the sealing plate 6 and the inner wall of the injection tube 5 is greater than the friction force between the rectangular block 32 and the inner wall of the rectangular groove 31, the rectangular block 32 on the puncture needle is enabled to slide on the inner wall of the rectangular groove 31. When the rectangular block 32 moves onto the bottom inner wall of the rectangular groove 31, the injection needle 7 is exactly inserted into the skin of the patient, and the injection rod 4 drives the sealing plate 6 to move such that the sealing plate 6 slides on the inner wall of the injection tube 5. In the injection process, the third pushrod motor 25 is started up, the third pushrod motor 25 drives the moving rods 26 to move, the moving rods 26 drive the arc-shaped blocks 27 to move to takeout the alcohol swab 28 which is used for wiping the body of the patient just now and to change a new alcohol swab 28, and the second pushrod motor 12 is started up. After the injection needle 7 is pulled out from the skin of the patient, the alcohol swab 28 is exactly in contact with the part of the patient on which the injection is performed, so as to complete the treatment of stopping bleeding for the patient.

The embodiments described above are merely preferred embodiments of the present invention. However, the protection scope of the present invention is not limited thereto. All equivalent replacements or modifications made by any one skilled in the art according to the technical solution of the present invention and the concept of the present invention within the technical scope disclosed by the present invention shall be included in the protection scope of the present invention.

1. A multifunctional assistant treatment robot, comprising a top plate, wherein, the bottom of the top plate is provided with a first mounting groove into which a first pushrod motor is fixedly mounted with its output shaft being connected with a puncture needle, and the bottom of the top plate is welded with a second vertical plate, the bottom of the top plate is further provided with an inclined groove, an inclined plate is slidably connected onto a top inner wall of the inclined groove, the first pushrod motor is located between the second vertical plate and the inclined plate, a treatment bed is provided below the first mounting groove, and a foam plate is placed at the top of the treatment bed and is located below the puncture needle.

2. The multifunctional assistant treatment robot of claim 1, wherein, the puncture needle comprises an injection rod, an injection tube and an injection needle, the output shaft of the first pushrod motor is connected with the injection rod, the bottom of the injection rod extends into the injection tube and is welded with a sealing plate which is slidably connected with an inner wall of the injection tube, an inner wall of one side of the inclined groove away from the first mounting groove is provided with a second mounting groove into which a second pushrod motor is fixedly mounted with its output shaft being welded with a pushing plate, one side of the inclined plate away from the first mounting groove is provided with a sliding groove, one side of the pushing plate close to the first mounting groove extends into the sliding groove and is slidably connected with inner walls of the sliding groove, inner walls of two sides of the sliding groove are provided with rectangular grooves, two sides of the pushing plate are welded with sliding blocks which are slidably connected with inner walls of the rectangular grooves, the bottom of the inclined plate is welded with a first vertical plate, the bottom of the first vertical plate extends below the top plate and is welded with a first horizontal plate, one side of the bottom of the first horizontal plate close to the first pushrod motor is provided with a third mounting groove into which a driving motor is fixedly mounted with its output shaft being welded with a rotating shaft, a bottom end of the rotating shaft is welded with two symmetrically arranged fixed plates, the sides of the two fixed plates close to each other are provided with a fourth mounting groove into which a third pushrod motor is fixedly mounted with its output shaft being welded with two moving rods, the ends of the two moving rods close to each other are welded with arc-shaped blocks, the sides of the two arc-shaped blocks close to each other are in contact with each other, and a same alcohol swab is fixedly mounted on inner sides of the two arc-shaped blocks and is located above the foam plate.

3. The multifunctional assistant treatment robot of claim 1, wherein, the bottom of the second vertical plate is welded with a second horizontal plate, one side of the second horizontal plate close to the puncture needle is provided with a rectangular groove, one side of the puncture needle close to the second horizontal plate is welded with a rectangular block which is slidably connected with an inner wall of the rectangular groove, the bottom of the second horizontal plate is provided with a fifth mounting groove in which a fourth pushrod motor is mounted with its output shaft being welded with a pressing plate, and the bottom of the pressing plate is welded with a massage plate which is located above the foam plate.

4. The multifunctional assistant treatment robot of claim 2, wherein, the arc-shaped blocks are located on one side of the puncture needle away from the second vertical plate, and the arc-shaped blocks are located above the foam plate.

5. The multifunctional assistant treatment robot of claim 2, wherein, the two arc-shaped blocks are symmetrically arranged, the sides of the two arc-shaped blocks close to each other are provided with sealing gaskets, and the alcohol swab is slidably connected with inner sides of the sealing gaskets.

6. The multifunctional assistant treatment robot of claim 2, wherein, the friction force between the sealing plate and the inner wall of the injection tube is greater than the friction force between the rectangular block and the inner wall of the rectangular groove.

7. The multifunctional assistant treatment robot of claim 3, wherein, the bottom of the second horizontal plate is further provided with a sixth mounting groove in which a music player is fixedly mounted, and the sixth mounting groove is located on one side of the fifth mounting groove away from the puncture needle.

8. The multifunctional assistant treatment robot of claim 3, wherein, the top of the fourth pushrod motor is welded with a mounting plate, four corners of the bottom of the mounting plate are provided with bolt holes into which bolts are threadedly connected, and the fourth pushrod motor is fixedly mounted on a top inner wall of the fifth mounting groove through the bolt holes and the bolts on the mounting plate.
